(19) United States
(12) Patent Application Publication
(10) Pub. No.: US 2004/0043975 A1
(43) Pub. Date: Mar. 4, 2004

(54) Topical application of alpha-DFMO and topical steroid for treatment of actinic keratoses

(75) Inventors: David S. Alberts, Tucson, AZ (US); Robert T. Dorr, Tucson, AZ (US)

Correspondence Address: Marvin A. Glazer, Esq., CAHILL, von HELLENS & GLAZER P.L.C., 155 Park One, 2141 East Highland Avenue, Phoenix, AZ, 85016, US

(73) Assignee: TOPICAL TECHNOLOGIES, INC.

(21) Appl. No.: 10/655,086

(22) Filed: Sep. 3, 2003

Related U.S. Application Data
(62) Division of application No. 10/041,236, filed on Jan. 7, 2002, now abandoned.

(51) Int. Cl. A61K 31/573

(52) U.S. Cl. 514/179

(57) ABSTRACT

Topical alpha-DFMO is mixed with a hydrophillic ointment base, along with at least one additional active drug, for treating actinic keratoses by topical application to human skin tissues. In one case, the topical steroid triamcinolone is combined with the alpha-DFMO. In a second case, the topical non-steroid anti-inflammatory diclofenac is combined with the alpha-DFMO. In a third instance, both triamcinolone and diclofenac are combined with the alpha-DFMO. In all such instances, topical application of such combinations was found to inhibit squamous cell cancer, and the combined effect of such components, when selected in appropriate proportions, in inhibiting squamous cell cancer cells is significantly greater than the effectiveness of each such component by itself.

### CROSS-REFERENCE TO RELATED APPLICATIONS

This patent application is a divisional application based upon co-pending U.S. patent application Ser. No. 10/041,236, filed Jan. 7, 2002, and the benefit of such earlier filing date is hereby claimed by Applicants under 35 U.S.C. §120.

### STATEMENT REGARDING FEDERALLY-SPONSORED RESEARCH

This invention was made with government support under Grant No. CA-27502 awarded by the National Institutes of Health (NIH) of Bethesda, Md. to the University of Arizona. The U.S. Government has a paid-up license in this invention.

### BACKGROUND OF THE INVENTION

1. Field of the Invention

The present invention relates generally to the treatment of actinic keratoses, and more particularly, to novel combinations of therapeutic agents for arresting the progression of actinic keratoses to squamous cell cancer, and for reducing the rate of squamous cell tumor growth.

2. Description of the Relevant Art

It is well-documented that squamous cell carcinoma and basal cell carcinoma typically develop within or adjacent to areas of pre-existing pre-malignant actinic keratoses (AKs). The presence of AKs represents a major risk factor for skin cancers. There is strong evidence that the incidence of skin cancer is increasing throughout the United States and other countries, particularly in regions closer to the equator where sunlight is more intense. Incidence rates for skin cancer are expected to increase further as the population ages and larger amounts of UV radiation reach the surface of the earth. Although the mortality rate for these skin cancers is low, their treatment is associated with considerable morbidity and remarkably high medical costs.

The most common treatments of AKs continues to be the topical application of 5% fluorouracil cream, or liquid nitrogen. Both of these methods result in severe inflammation, erythema, and superficial ulceration. There continues to be a need for the development of less toxic drugs which can be applied chronically as chemopreventive agents for patients with severely sun-damaged skin and AKs.

DFMO is an enzyme-activated irreversible inhibitor of ornithine decarboxylase, which is the rate-limiting enzyme in polyamine synthesis, and decreases intracellular levels of putrescine and spermidine in the skin and other vital tissues. In conjunction with the administration of model carcinogens, DFMO has been shown to significantly reduce tumor incidence in several mammalian in vivo tests for chemopreventive activity. Additionally, DFMO chemopreventive activity has been demonstrated in chemical and UV models of mouse skin carcinogenesis. It has also been shown that p.o.-administered DFMO reduced UVB-induced skin cancers in C3H/HeN mice from 38% in placebo treated controls to 9% in treated animals. Similarly, topically administered DFMO in an acetone vehicle dramatically reduced UVB-induced skin cancers in BALB/c mice. In adult participants with psoriasis, the application of 10% DFMO cream resulted in a 66% reduction in spermidine concentrations in the skin and a marginal improvement in psoriatic lesions.

Within U.S. Pat. No. 5,851,537, applicants disclosed the topical application of alpha-DFMO in a hydrophilic cream salve carrier for use as a topical chemopreventive agent against skin cancer. As set forth in such patent, tests on human subjects showed that the topical application of such cream reduced the number of AKs on the arms of such subjects. However, several of the subjects experienced skin rashes in the area where the alpha-DFMO was applied.

Skin inflammation has also been noted as a negative side effect when other methods of treating actinic keratoses have been used. For example, it has already been mentioned that fluorouracil has been applied topically to treat AKs. Back in 1976, Breza, et al., “Noninflammatory Destruction of Actinic Keratoses by Flourouracil”, Arch Dermatol, Vol. 112, September 1976, pp. 1256, proposed that 0.5% triamcinolone acetonide cream be added to topical compositions of fluorouracil in order to suppress the inflammatory reaction associated with the topical fluorouracil therapy of actinic keratoses. Breza, et al. concluded that the addition of 0.5% triamcinolone acetonide did not alter the effectiveness of the fluorouracil, but that it did serve to reduce inflammatory response.

Diclofenac is a topical NSAID (non-steroidal anti-inflammatory drug), and has been used in the past for the treatment of pain and inflammation in rheumatoid arthritis by inhibiting cyclooxygenase enzymes. More recently, the FDA has approved the drug diclofenac in topical gel form, available from Skylabs/SkyePharma Inc. of San Diego, Calif. and/or its British affiliate, under the trade designation “Solareze”, for treatment of actinic keratoses. It belongs to the family of medicines called antineoplastics, at least some of which are believed to kill cancerous cells.

While some of the compositions mentioned above have been shown to have varying degrees of effectiveness in treating actinic keratoses, no evidence known to the applicants has suggested that any of such treatment methods is actually effective to arrest or reduce tumor growth of actual squamous cell carcinoma and basal cell carcinoma tumors.

In view of the foregoing, it is an object of the present invention to provide a topical composition and method for treating actinic keratoses which is more effective than any of the known treatment methods described above.

It is also an object of the present invention to provide such a topical composition and method that is less toxic than known treatment methods described above.

Another object of the present invention is to lessen the likelihood of skin rashes when alpha-DFMO is used to treat actinic keratoses in humans.

Yet another object of the present invention is to enhance the effectiveness of alpha-DFMO when topically treating actinic keratoses in humans.

Still another object of the present invention is to enhance the effectiveness of the drug diclofenac when topically treating actinic keratoses in humans.

It is a further object of the present invention to provide a topical composition and method for treating actinic keratoses using reduced concentrations of alpha-DFMO without compromising the effectiveness of the treatment.

Still a further object of the present invention is to provide a topical composition and method that are effective to inhibit and/or reduce the growth rate of existing skin cancers.

These and other objects of the present invention will become more apparent to those skilled in the art as the description of the present invention proceeds.

### SUMMARY OF THE INVENTION

Briefly described, and in accordance with one embodiment of the thereof, the present invention relates to a method for treating actinic keratoses by topical application to the skin tissues of a human being containing actinic keratoses lesions. In practicing such method, alpha-DFMO and a topical steroid, preferably topical triamcinolone (a corticosteroid), are combined with a base vehicle such as a hydrophilic ointment (Hydrophilic Ointment, USP), Vanicream® topical vanishing-cream, or the like; such base vehicles can include both water-in-oil emulsions and oil-in-water emulsions. Though testing is yet to be conducted, applicants believe that other topical steroidals may be substituted for topical triamcinolone, including betamethasone, clobetasol, dexamethasone, furoate, fluocinonide, amcinonide, desonide, desoximetasone, fluocinolone, fluticasone, halobetasol, hydrocortisone, and mometasone. This combination is applied topically to the skin of a human being having actinic keratoses lesions in order to reduce the number of such actinic keratoses lesions and to reduce spermidine concentrations associated with such skin tissues. Preferably, topical alpha-DFMO represents from 0.1% to 20% by weight of the applied combination. It is also preferred that topical triamcinolone be present within the range of 0.001% to 1.0% by weight. Preferred relative ratios by weight of alpha-DFMO to triamcinolone combined with the base vehicle ranges between 0.1:2 and 40:1.

The present invention also relates, in conjunction with another embodiment, to a method for treating actinic keratoses by topical application to the skin tissues of a human being containing actinic keratoses lesions, and using a three-drug combination within the base vehicle. This method includes the steps of combining alpha-DFMO, a topical steroid, preferably triamcinolone, and a topical non-steroid anti-inflammatory, preferably diclofenac, to the base vehicle, and applying the combination to the skin of a human being having actinic keratoses lesions in order to reduce the number of such actinic keratoses lesions and to reduce spermidine concentrations associated with such skin tissues. Other topical non-steroid anti-inflammatory drugs which may be used in place of diclofenac include difunisal, etodolac, fenoprofen, ketoprofen, ketorolac, mefenamic acid, nabumetone, naproxen, oxaprozin, tolmetin sodium, ibuprofen, celecoxib, rofecoxib, choline salicylate, and sodium salicylate. Preferably, the alpha-DFMO is present within the range of 0.1% to 20% by weight; likewise, in the preferred embodiment, the topical diclofenac has a concentration within the range of 0.1%-10% by weight. The relative ratio of alpha-DFMO to topical diclofenac, by weight, within the preferred embodiment, lies between 1:10 and 200:1. Again, the base vehicle is preferably a hydrophilic ointment (Hydrophilic Ointment, USP), Vanicream® topical vanishing cream, a water-in-oil emulsion, an oil-in-water emulsion, or the like.

### BRIEF DESCRIPTION OF THE DRAWINGS

FIG. 1 is a graph illustrating the comparative impact of using alpha-DFMO alone, versus alpha-DFMO plus trianicinolone, in reducing the survival rate of I-7 human ras-transfected epidermal keratinocytes.

FIG. 2 is a table setting forth test data showing the relative effects of using alpha-DFMO alone, triamcinolone alone, and combinations of alpha-DFMO plus triamcinolone, in varying combinations, in order to reduce the survival rate of I-7 human ras-transfected epidermal keratinocytes.

FIG. 3 is a graph illustrating the comparative impact of using alpha-DFMO alone, versus alpha-DFMO plus triamcinolone, in reducing the survival rate of 11-4 human squamous skin cancer cells.

FIG. 4. is a table setting forth test data showing the relative effects of using alpha-DFMO alone, triamcinolone alone, and combinations of alpha-DFMO plus triamcinolone, in varying combinations, in order to reduce the survival rate of 11-4 human squamous skin cancer cells.

FIG. 5 is a graph illustrating the combined effect of topical alpha-DFMO and triamcinolone on human II-4 squamous skin cancer cells injected into SCID mice.

FIG. 6 is a graph illustrating the impact of alpha-DFMO and triamcinolone, alone and in combination, upon growth rates of human squamous cell skin cancer volume.

FIG. 7 is a data table showing the results of an in vitro evaluation of the topical combination of alpha-DFMO, triamcinolone, and diclofenac upon the growth of II-4 human squamous skin cancer cells.

### DETAILED DESCRIPTION OF THE PREFERRED EMBODIMENTS

As mentioned above, alpha-DFMO is an enzyme-activated irreversible inhibitor of ornithine decarboxylase, and it acts to decrease intracellular levels of putrescine and spermidine in the skin. For purposes of the practice of the present invention, alpha-DFMO was formulated by starting with a white powder of the monohydrate, monochloride form, having a molecular weight of 236.65, commercially available originally from Marrion-Merrell Dow Pharmaceutical Company of Kansas City, Mo., and now available from Sigma Chemicals, St. Louis, Mo. for in vitro and in vivo mouse studies, and from ILEX Oncology, San Antonio, Tex. for clinical studies. Such alpha-DFMO is mixed with a base vehicle for topical application. The preferred base vehicle is a hydrophilic ointment (Hydrophilic Ointment, USP) commercially available from E. Fougera & Company of Melville, N.Y.

For purposes of practicing the present invention, triamcinolone (triamcinolone acetonide) was of the type formulated by E. Fougera & Company of Melville, N.Y. Appropriate amounts of the alpha-DFMO and/or triamcinolone were weighed-out and mixed in a blender with the hydrophilic ointment. Once mixed, the combination was transferred to polyethylene-lined, 30 gram metal ointment tubes which were crimp-sealed to preclude exposure to light and air. In some studies, control groups received pure hydrophilic ointment applied topically, or no treatment at all.

It was found that the addition of the topical steroid reduces alpha-DFMO induced inflammatory response in the skin. As mentioned above, topical steroids have been used in the past to reduce skin inflammation. Surprisingly, however, the in vitro addition of the topical steroid under cell culture conditions has been found to significantly enhance the effectiveness of alpha-DFMO in reducing the growth of immortalized human keratinocytes and ras-transfected human squamous cell keratinocytes. Additionally, the addition of the topical steroid has been found to significantly enhance the effectiveness of topical alpha-DFMO in reducing squamous cell skin tumors implanted in immunodeficient mice. In other words, the combination of the topical steroid triamcinolone with topical alpha-DFMO has shown an unpredictable synergistic effect relative to reduction of squamous cell skin tumors.

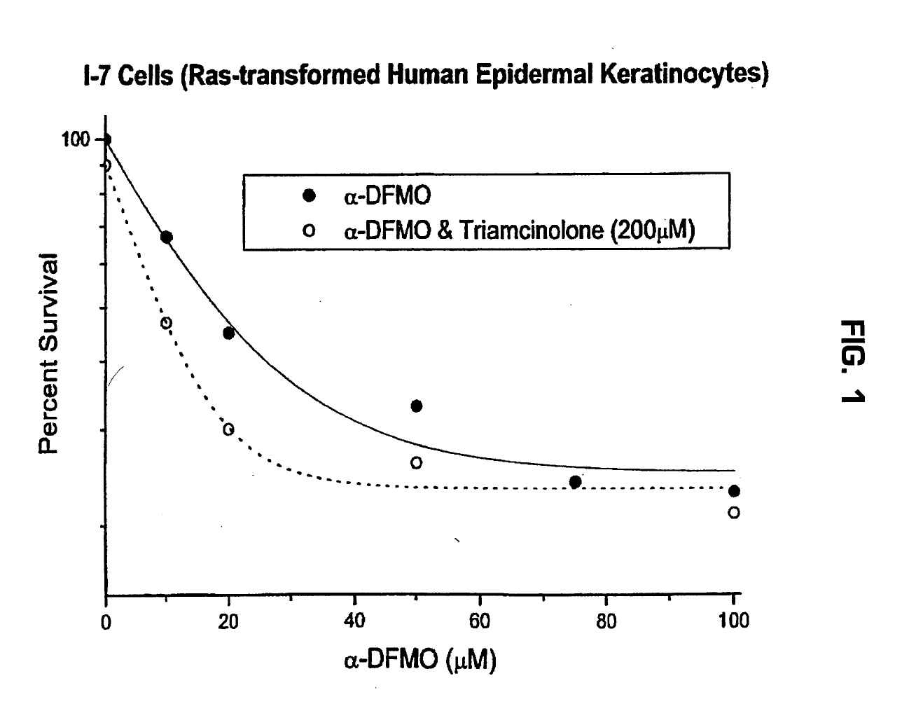

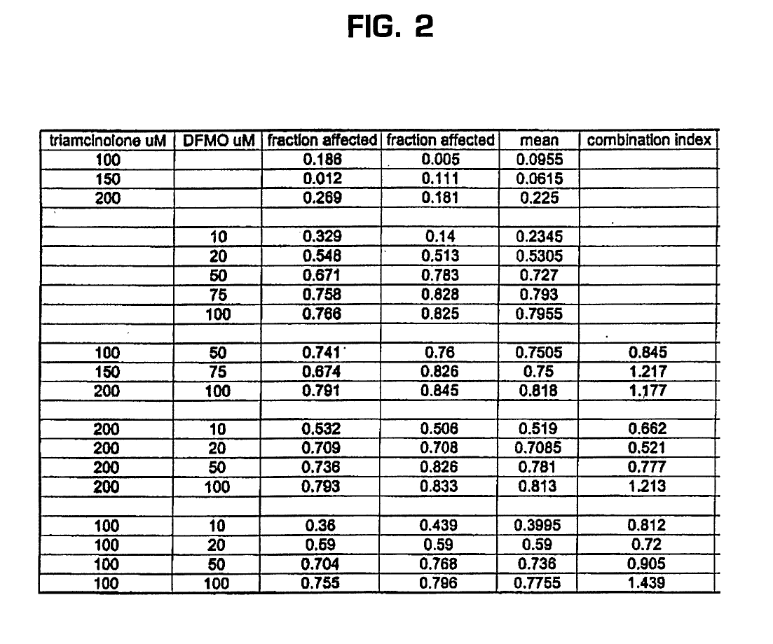

One method used to demonstrate the unexpected effectiveness of the combination of the topical steroid triamcinolone with topical alpha-DFMO was an in-vitro study performed in a laboratory using two different human cutaneous cell lines transfected with a mutated ras gene. The Median Dose Effect Principal Method was used to evaluate additivity and synergism of the two components alpha-DFMO triamcinolone and triamcinolone. The graph set forth in FIG. 1 shows the percent survival rate of Type I-7 transformed keratinocyte cells as a function of the microMolar concentration of the alpha-DFMO. The FIG. 1 graph demonstrates that, for concentrations of alpha-DFMO in the range of approximately 10 to 60 microMolar, the addition of 200 microMolar triamcinolone significantly improved the effectiveness of alpha-DFMO alone relative to the reduction of Type I-7 actinic keratoses cells. All of the data recorded for Type I-7 actinic keratoses cells is set forth in the data table of FIG. 2.

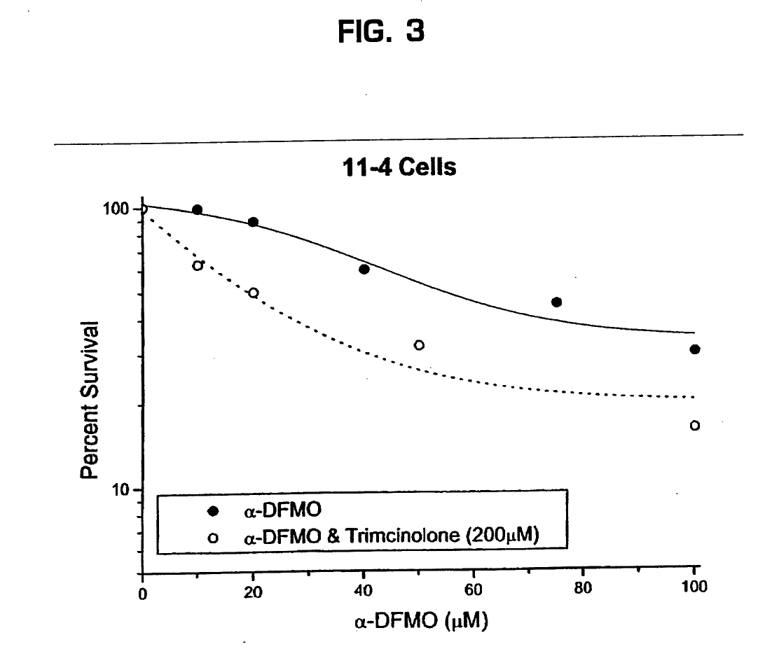

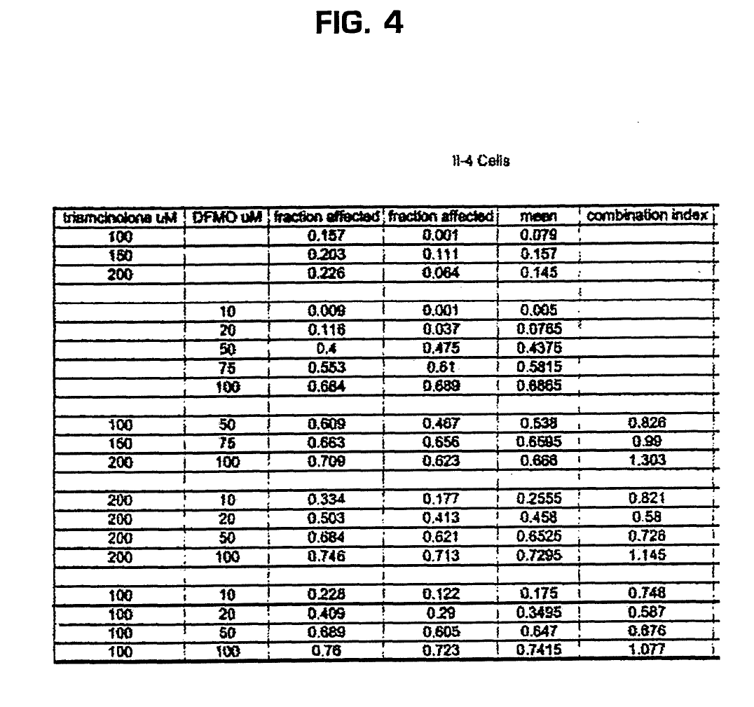

Likewise, the graph set forth in FIG. 3 shows the percent survival rate of Type II-4 squamous cancer skin cells as a function of the microMolar concentration of the alpha-DFMO. The FIG. 3 graph demonstrates that, for concentrations of alpha-DFMO in the range of approximately 10 to 100 microMolar, the addition of 200 microMolar triamcinolone significantly improved the effectiveness of alpha-DFMO alone relative to the reduction of Type II-4 squamous cancer skin cells. All of the data recorded for Type II-4 squamous cancer skin cells is set forth in the data table of FIG. 4.

In a second study, in vivo tests were made using laboratory mice, along with the II-4 human squamous skin cell line. Human keratinocytes were modified for immortalization by transfection of the c-Harvey-Ras (EJ) oncogene, pursuant to the methods described in Boukamp, et al., “c-Ha-ras Oncogene expression in immortalized human keratinocytes (HaCaT) Alters growth potential in vivo but lacks correlation with malignancy”, Cancer Research, 50:2840-2847, 1990, the contents of which are hereby incorporated by reference. This skin squamous cancer cell line, termed HaCaT II-4, has been shown to produce malignant tumor growth when injected subcutaneously into immunologically deficient (SCID) mice. The tumors that form are invasive, but still form an epidermis-like stratified epithelium when transplanted.

In this second study, the topical formulation that was tested consisted of a 10% (w/w) of alpha-DFMO added to 0.1% (w/w) triamcinolone acetonide, along with a topical vehicle. The 10% alpha-DFMO was originally obtained from Marion Merrell, Dow Corp., which later merged into Hoecsht Pharmaceuticals, which licensed all manufacturing and intellectual property rights to ILEX Oncology, San Antonio, Tex. The 0.1% triamcinolone was obtained from Sigma Chemical of St. Louis, Mo. The topical vehicle selected was a Hydrophilic Ointment, USP, obtained from E. Fougera & Co., of Melville, N.Y., although Vanicreame® topical vanishing cream or like vehicles can also be used. The powdered drugs were mixed in a blender into the ointment base and stored for later use at room temperature.

The mice used for the second study were severe-combined immune deficiency (SCID) mice provided from the Arizona Cancer Center breeding facility. Sixteen of such mice were divided into four groups in the manner explained below. The tumor cells were initially grown in vitro in sterile cell culture in sufficient quantity to inject each mouse with 10 million cells; these cells were injected subcutaneously into the front flank muscle on Day 0 of the study. The relative time at which topical treatment was started was varied among three groups of mice; one group of mice received pre-treatment starting ten days before tumor implantation, a second group of mice began receiving topical treatment one day after tumor implantation (before any tumor was palpable), and a third group of mice began receiving treatment eight days after tumor implantation (when a mean 30 mm³ tumor was palpable). A fourth group of mice served as a control group, and were treated with pure hydrophilic ointment, lacking any alpha-DFMO or triamcinolone, beginning the first day after tumor implantation. Topical treatment consisted of the application of 100 μL of the above-described ointment mixture delivered by positive-displacement pipette daily. The control group received an equal amount of pure hydrophilic ointment.

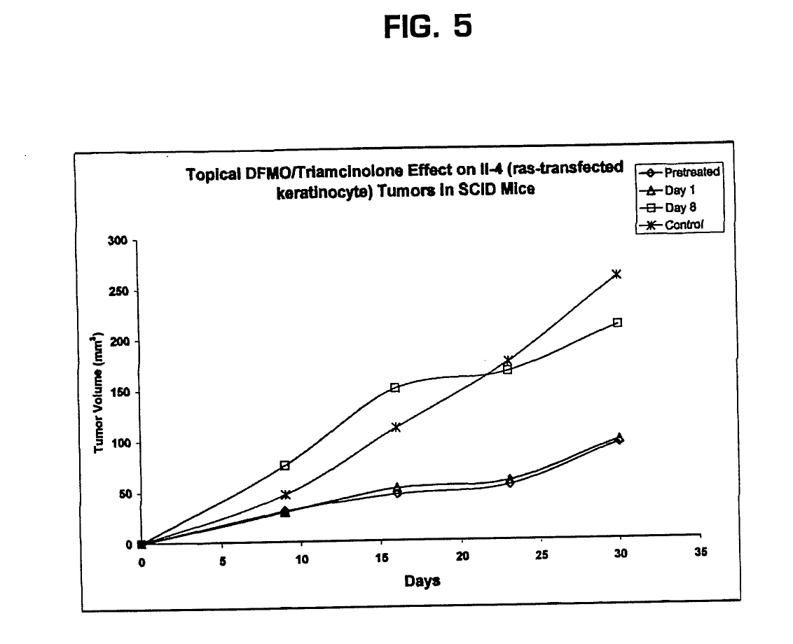

The results of this second study are depicted within the graph of FIG. 5. Significant anti-tumor activity (reduced rate of tumor growth) was observed in both the first (pre-treatment) and second (treatment beginning on Day 1) groups of mice as compared with the control group. However, the third group of mice (treatment delayed until Day 8) did not demonstrate significant tumor growth inhibition as compared with the control group. Within this second study, there was no visual evidence of local toxicity at the application site of the topical treatment at any time during the study.

This second study indicated that the combination of topical alpha-DFMO plus triamcinolone was tolerated when applied topically to SCID mice. The results of the second study further demonstrate that when the combination of topical alpha-DFMO plus triamcinolone is applied topically in appropriate amounts, prior to the development of a palpable tumor mass, such treatment inhibits growth of a human keratinocyte-derived squamous cell cancer.

In a third study of the effects of combining alpha-DFMO and triamcinolone, SCID mice (4/group) were given 10⁷ II-4 cells and then randomized to receive topical treatment with triamcinolone acetonide (0.05% or 0.1%), α-DFMO, (5% or 10%), or the combination of α-DFMO with triamcinolone. The control groups received either no treatment (n=4), or the ointment base (hydrophilic ointment, U.S.P. alone (n=4), or the ointment base (hydrophilic ointment, U.S.P. alone (n=4). Treatments were given daily to the tumor-bearing blank skin continuously beginning 24 hours after tumor implantation. Palpable tumors were measured bi-dimensionally using calipers, three times per week, until tumor sizes approximated 1,500 mm³ (about 1.5 g). The mice were then euthanized.

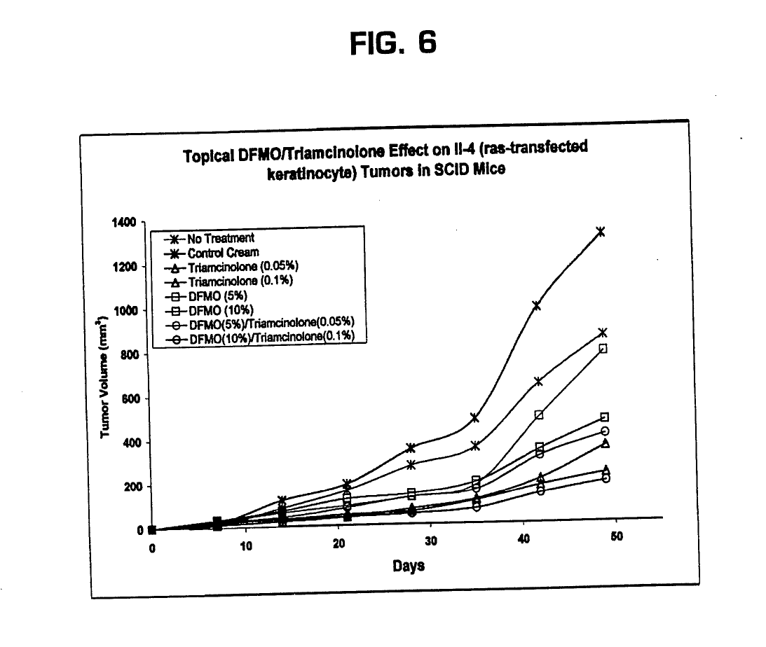

The results of this third study are shown in the graph of FIG. 6. In the graph of FIG. 6, tumor cell volume is plotted as a function of time over a period of fifty days for no treatment; hydrophilic ointment only; 5% and 10%, respectively, alpha-DFMO only; 0.05% and 0.1%, respectively, of triamcinolone only; and the combinations of 5% alpha-DFMO/0.05% triamcinolone, and 10% alpha-DFMO/0.1% triamcinolone. The graph of FIG. 6 shows that tumor cell volume grew the fastest either when there was no treatment, or when the topical application consisted merely of hydrophilic ointment. The rate of growth for hydrophilic ointment alone is actually faster than for no treatment at all, indicating that the rubbing of such tumor cells through topical application of the ointment actually stimulates tumor growth. The graph of FIG. 6 further illustrates that the application of alpha-DFMO by itself decreases the rate of tumor growth, and that the application of triamcinolone by itself decreases the rate of tumor growth. Finally, the graph of FIG. 6 demonstrates that the tumor growth rate was reduced the most by the combination of 10% (by weight) alpha-DFMO and 0.1% triamcinolone.

Thus, topical application of alpha-DFMO plus triamcinolone not only inhibits potentially-cancerous actinic keratoses cells from becoming cancerous (chemo-prevention of tumor development), but actually reduces the number of cells that have already become cancerous (inhibition of frank tumor growth). Relative concentrations of topical alpha-DFMO believed to be effective lie within the range of 0.1% to 20% by weight. Likewise, relative concentrations of topical triamcinolone which are believed to be effective lie within the range of 0.001% to 1.0% by weight. Preferred relative ratios by weight of alpha-DFMO to triamcinolone combined with the base vehicle ranges between 0.10:2 and 40:1.

The applicants conducted a further study to investigate the effects of combining topical alpha-DFMO with topical diclofenac (a topical non-steroid anti-inflammatory) as a method for treating actinic keratoses by topical application to the skin tissues of a human being containing actinic keratoses lesions. In this study, the effect of such combination was investigated in regard to II-4 squamous cancer cells. In preparing the topical application for the study, the same hydrophilic ointment mentioned above was used as a base vehicle. Alpha-DFMO in the amount of 5% (w/w) by weight was added to the base vehicle. In addition, 1% (w/w) by weight diclofenac was combined therewith. The resulting combination was applied to SCID mice given 10⁷ II-4 cells in the flank one day previously.

Within the article by Alberts, et al., “Pharmacologic Studies of Anticancer Drugs with the Human Tumor Stem Cell Array”, Cancer Chemother Pharmacol, 1981, 6:253-264, the contents of which are hereby incorporated by reference, an explanation is provided for assessing the treatment efficacy of combining two or more drugs. The method described in such article is based upon the so-called “Fractional Survival Method of Drewinko, et al.”. This article describes the difference between combinations which are merely additive, combinations which are antagonistic, and combinations that render truly synergistic results.

The test results obtained from the above-described combination of topical alpha-DFMO with topical diclofenac indicated that the experimentally-obtained fractional survival effect of such combination was significantly improved over the fractional survival effect that would have been predicted algebraically by simply multiplying together the individual fractional survival effects of alpha-DFMO and diclofenac when used alone. The preferred concentration of topical diclofenac is within the range of 0.1%-10% by weight, while the alpha-DFMO has a preferred concentration within the range of 0.10% to 20% by weight. The relative ratio by weight of alpha-DFMO to topical diclofenac combined with the base vehicle preferably ranges between 2:1 and 20:1. While the aforementioned study used diclofenac as the topical non-steroid anti-inflammatory, other suitable non-steroidal anti-inflammatory compounds include ketoprofen, ibuprofen, celecoxib, salicylate, difunisal, etodolac, fenoprofen, ketorolac, mefenamic acid, nabumetone, naproxen, oxaprozin, tolmetin sodium, and rofecoxib.

A further in vitro study was conducted by the applicants to investigate the efficacy of the topical application of alpha-DFMO, triamcinolone, and diclofenac as a method for treating actinic keratoses by topical application to the skin tissues of a human being. Alpha-DFMO and triamcinolone were added together with diclofenac. This study was directed to II-4 human squamous cell skin cancer cells.

The cell culture was maintained as a monolayer culture. The cells were plated in 96 well microtiter plates on Day 0 of the study. Some of such wells were used as control wells, so no drugs were added thereto. The aforementioned combination of drugs was added to the microtiter test wells on Day 1. On Day 7, the plates were fixed with cold 10% trichloroacetic acid, and then washed four times with distilled water. Each plate was then stained with 0.4% sulforhodamine B. Excess stain was then removed by washing the plate four times with 1% acetic acid. The stained dye is then solubilized with 50 mM Tris. The stained plates were then “read”, i.e., measured for optical density, on an automatic plate reader at 540 nm. A lower optical density corresponds to a smaller volume of cancerous cells, and a higher optical density corresponds to a larger volume of cancerous cells. A surviving fraction factor fₛ is then computed for the treated well plates by taking the mean optical density of the treated well plates and dividing by the mean optical density of the control well plates.

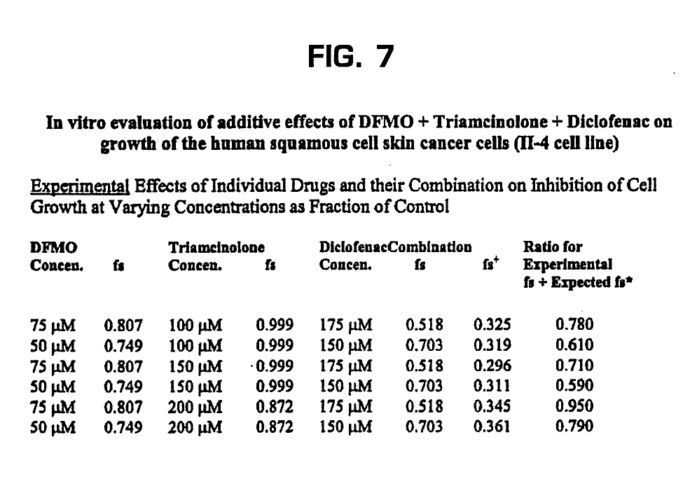

The results of this in vitro study on the three-drug combination are set forth in the table of FIG. 7. Within the first two columns of FIG. 7, the individual surviving fraction factors fₛ (0.749 and 0.807) are set forth for two different concentrations (50 μM and 75 μM, respectively) of alpha-DFMO. While a lower surviving fraction factor 0.749 is associated with the smaller dosage of 50 μM, and a higher surviving fraction factor 0.807 is associated with the greater dosage of 75 μM, the difference in such numbers is probably not statistically significant. Likewise, in the second pair of columns of the table of FIG. 7, the individual surviving fraction factors fₛ(0.999, 0.999 and 0.872) are set forth for three different concentrations (100 μM, 150 μM, and 200 μM, respectively) of triamcinolone. It has been noted that the surviving fraction factor 0.999 is the same for both 100 μM and 150 μM, whereas increasing the dosage from 150 μM to 200 μM produces a noticeable improvement; this could be due to a threshold effect that is overcome at doses exceeding 150 μM. The third pair of columns in the table of FIG. 7 show the individual surviving fraction factors fₛ(0.703 and 0.518) for two different concentrations (150 μM and 175 μM, respectively) of diclofenac. The column entitled fₛ+ shows the actual surviving fraction factor measured in the laboratory for each such three-drug combination.

As explained in the aforementioned article by Alberts, et al., a mathematically-derived expected fraction factor (fₛ*) can be computed for each three-drug combination by multiplying together the individual fraction factors for the three individual drugs. A ratio can then be computed for the actual experimentally-determined fraction factor fₛ divided by the expected fraction factor fₛ*, and this ratio is presented in the rightmost column of the table in FIG. 7. As further explained in the article by Alberts, et al., a ratio of 1:1 indicates that a drug combination is additive, whereas a ratio of less than 1:1 indicates that the drug combination is synergistic. The lower the ratio, the more synergistic is the combination. As will be noted, all of the ratio values in the rightmost column of the table of FIG. 7 are less than 1:1, with at least two of the combinations producing ratios as low as approximately 0.6:1.

Those skilled in the art will now appreciate that an improved topical composition and method for treating actinic keratoses has been described which is highly effective in topically treating actinic keratoses in humans. The disclosed composition and method is less toxic than known treatment methods, and lessens the likelihood of skin rashes when alpha-DFMO is used to treat actinic keratoses in humans. Indeed, in past studies, when alpha-DFMO was used alone without a topical steroid or non-steroidal anti-inflammatory, there was approximately a 20% incidence of skin reactions; in contrast, studies conducted to date for the combination of alpha-DFMO plus triamcinolone show no topical hypersensitivity reactions. The disclosed method unexpectedly enhances the effectiveness of both alpha-DFMO and diclofenac when topically treating actinic keratoses in humans. Perhaps most importantly, the compositions and methods of the present invention are effective not only to prevent pre-malignant actinic keratoses from progressing to malignant squamous cell cancer, but also to inhibit and/or reduce the growth rate of existing squamous cell skin cancer.

While the present invention has been described with respect to preferred embodiments thereof, such description is for illustrative purposes only, and is not to be construed as limiting the scope of the invention. The generic name for alpha-DFMO is eflornithine, which is a racemic mixture of two enantiomers. It has been theorized that one of such enantiomers may be associated with a slight risk of ototoxicity (hearing loss), though Applicants do not believe that any systemic toxicity results from the topical application of racemic topical alpha-DFMO because there was negligible systemic uptake in animal studies, and no drug was detected in clinical trials following topical application of alpha-DFMO. Nonetheless, it may be possible to obtain the advantages of the present invention as described above using only one or the other of the two enantiomers that form the racemic mixture of alpha-DFMO; accordingly, the use of the term “alpha-DFMO” herein should be understood, for purposes of the present patent application, to include both the racemic mixture of the two enantiomers that normally make up alpha-DFMO, as well as each of the two enantiomers that collectively make up the racemic mixture of alpha-DFMO. Various modifications and changes may be made to the described embodiments by those skilled in the art without departing from the true spirit and scope of the invention as defined by the appended claims.

We claim:

1. A method for treating actinic keratoses by topical application to the skin tissues of a human being containing actinic keratoses lesions, said method comprising the steps of:
a. providing a base vehicle;
b. combining alpha-DFMO to the base vehicle;
c. combining a topical steroid to the base vehicle;
d. applying the combination formed in steps a, b, and c to the skin of a human being having actinic keratoses lesions in order to reduce the number of such actinic keratoses lesions and to reduce spermidine concentrations associated with such skin tissues.

2. The method recited by claim 1 wherein the topical steroid combined in step c is topical triamcinolone.

3. The method recited by claim 2 wherein the topical triamcinolone has a relative concentration within the range of 0.001% to 1.0% by weight.

4. The method recited by claim 2 wherein the alpha-DFMO has a concentration within the range of 0.1% to 20% by weight.

5. The method recited by claim 4 wherein the topical triamcinolone has a concentration within the range of 0.01 to 1.0% by weight.

6. The method recited by claim 2 wherein the relative ratio by weight of alpha-DFMO to triamcinolone combined with the base vehicle ranges between 0.10:2 and 40:1.

7. The method recited by claim 1 wherein the base vehicle is a hydrophilic ointment.

8. The method recited by claim 1 wherein the base vehicle is Vanicream® topical vanishing cream.

9. The method recited by claim 1 wherein the base vehicle is a water-in-oil emulsion.

10. The method recited by claim 1 wherein the base vehicle is an oil-in-water emulsion.

11. The method recited by claim 1 wherein the topical steroid is selected from the group of topical steroids consisting of triamcinolone, betamethasone, clobetasol, dexamethasone, furoate, fluocinonide, amcinonide, desonide, desoximetasone, fluocinolone, fluticasone, halobetasol, hydrocortisone, and mometasone.

12. The method recited by claim 1 including the further step of:
e. combining a topical non-steroid anti-inflammatory to the base vehicle prior to step d.

13. The method recited by claim 12 wherein the topical steroid combined in step c is topical triamcinolone.

14. The method recited by claim 13 wherein the topical non-steroid anti-inflammatory combined in step e is topical diclofenac.

15. The method recited by claim 12 wherein the topical non-steroid anti-inflammatory combined in step c is topical diclofenac.

16. The method recited by claim 12 wherein the topical steroid combined in step c is selected from the group of topical steroids consisting of triamcinolone, betamethasone, clobetasol, dexamethasone, furoate, fluocinonide, amcinonide, desonide, desoximetasone, fluocinolone, fluticasone, halobetasol, hydrocortisone, and mometasone.

17. The method recited by claim 12 wherein the topical non-steroid anti-inflammatory combined in step e is selected from the group of non-steroidal anti-inflammatory compounds consisting of diclofenac, difunisal, etodolac, fenoprofen, ketoprofen, ketorolac, mefenamic acid, nabumetone, naproxen, oxaprozin, tolmetin sodium, ibuprofen, celecoxib, rofecoxib, and choline salicylate.

18. A method for topically treating squamous skin cancer lesions by topical application to the skin tissues of a human being containing squamous skin cancer lesions, said method comprising the steps of:
a. providing a base vehicle;
b. combining alpha-DFMO to the base vehicle;
c. combining a topical steroid to the base vehicle;
d. applying the combination formed in steps a, b, and c to the skin of a human being having squamous skin cancer lesions in order to reduce the number of such squamous skin cancer lesions.

19. The method recited by claim 18 wherein the topical steroid combined in step c is topical triamcinolone.

20. The method recited by claim 19 wherein the topical triamcinolone has a relative concentration within the range of 0.001% to 1.0% by weight.

21. The method recited by claim 19 wherein the alpha-DFMO has a concentration within the range of 0.5% to 20% by weight.

22. The method recited by claim 21 wherein the topical triamcinolone has a concentration within the range of 0.01 to 1.0% by weight.

23. The method recited by claim 19 wherein the relative ratio by weight of alpha-DFMO to triamcinolone combined with the base vehicle ranges between 0.10:2 and 40:1.

24. The method recited by claim 18 wherein the base vehicle is a hydrophilic ointment.

25. The method recited by claim 18 wherein the base vehicle is Vanicream® topical vanishing cream.

26. The method recited by claim 18 wherein the base vehicle is a water-in-oil emulsion.

27. The method recited by claim 18 wherein the base vehicle is an oil-in-water emulsion.

28. The method recited by claim 18 wherein the topical steroid is selected from the group of topical steroids consisting of triamcinolone, betamethasone, clobetasol, dexamethasone, furoate, fluocinonide, amcinonide, desonide, desoximetasone, fluocinolone, fluticasone, halobetasol, hydrocortisone, and mometasone.
